(19) United States
(12) Patent Application Publication
(10) Pub. No.: US 2019/0276399 A1
(43) Pub. Date: Sep. 12, 2019

(54) POLYMORPH OF NINTEDANIB

(71) Applicant: Olon S.P.A., Rodano (MI) (IT)

(72) Inventors: Barbara Novo, Rodano (MI) (IT); Jacopo Bonanomi, Rodano (MI) (IT); Stella De Fiore, Rodano (MI) (IT); Francesco Calogero, Rodano (MI) (IT)

(73) Assignee: Olon S.P.A., Rodano (MI) (IT)

(21) Appl. No.: 16/304,927

(22) PCT Filed: Jun. 6, 2017
(86) PCT No.: PCT/EP2017/063640

§ 371 Date: Nov. 27, 2018

(30) Foreign Application Priority Data
Jun. 8, 2016 (IT) 102016000058795

(51) Int. Cl. C07D 209/34

(52) U.S. Cl. CPC C07D 209/34; C07B 2200/13

(57) ABSTRACT

Disclosed is a partly crystalline form of nintedanib ethanesulphonate and its preparation process.

### SUMMARY OF THE INVENTION

The present invention relates to a partly crystalline form of nintedanib ethanesulphonate and a process for its preparation.

### BACKGROUND TO THE INVENTION

Nintedanib ethanesulphonate (methyl-(3Z)-3-{[(4-{methyl[(4-methylpiperazin-1-yl) acetyl]amino}phenyl)amino](phenyl)methylidene}-2-oxo-2,3-dihydro-1H-indole-6-carboxylate) is a known tyrosine kinase inhibitor which exerts its activity on vascular endothelial growth factor receptors (VEGFR), fibroblast growth factor receptors (FGFR) and platelet-derived growth factor receptors (PDGFR). Nintedanib ethanesulphonate is used in the treatment of small-cell lung adenocarcinoma when the tumor is locally advanced, metastatic or locally recurrent. Nintedanib ethanesulphonate is used in combination with docetaxel in patients who have already undergone prior chemotherapy.

WO2004013099 describes a crystalline form of nintedanib ethanesulphonate which shows an XRPD spectrum (λ=1.5418 Å) containing the most intense peaks at 2θ=7.7, 8.7, 9.4, 9.8, 11.5, 11.9, 13.1, 13.7, 14.1, 16.3, 16.7, 16.9, 17.4, 17.7, 18.5, 18.8, 19.0, 20.0, 21.2, 21.7, 22.1, 22.5, 23.1, 23.8, 24.7, 24.8, 24.9, 25.4, 26.2, 26.9, 27.1, 27.6, 27.9, 28.7 and 29.2.

Particular crystalline forms of nintedanib ethanesulphonate can have advantageous properties in terms of their solubility and/or stability and/or bioavailability and/or impurity profile and/or filtration characteristics and/or drying characteristics and/or absence of hygroscopicity and/or their ability to be handled and/or micronized and/or preparation of tablets.

### DESCRIPTION OF THE FIGURES

FIG. 1: Infrared spectrum of the novel partly crystalline form of nintedanib ethanesulphonate.

FIG. 2: DSC curve of the novel partly crystalline form of nintedanib ethanesulphonate.

FIG. 3: XRPD pattern of the novel partly crystalline form of nintedanib ethanesulphonate.

FIG. 4: ¹H-NMR spectrum of the novel partly crystalline form of nintedanib ethanesulphonate in d₆-DMSO.

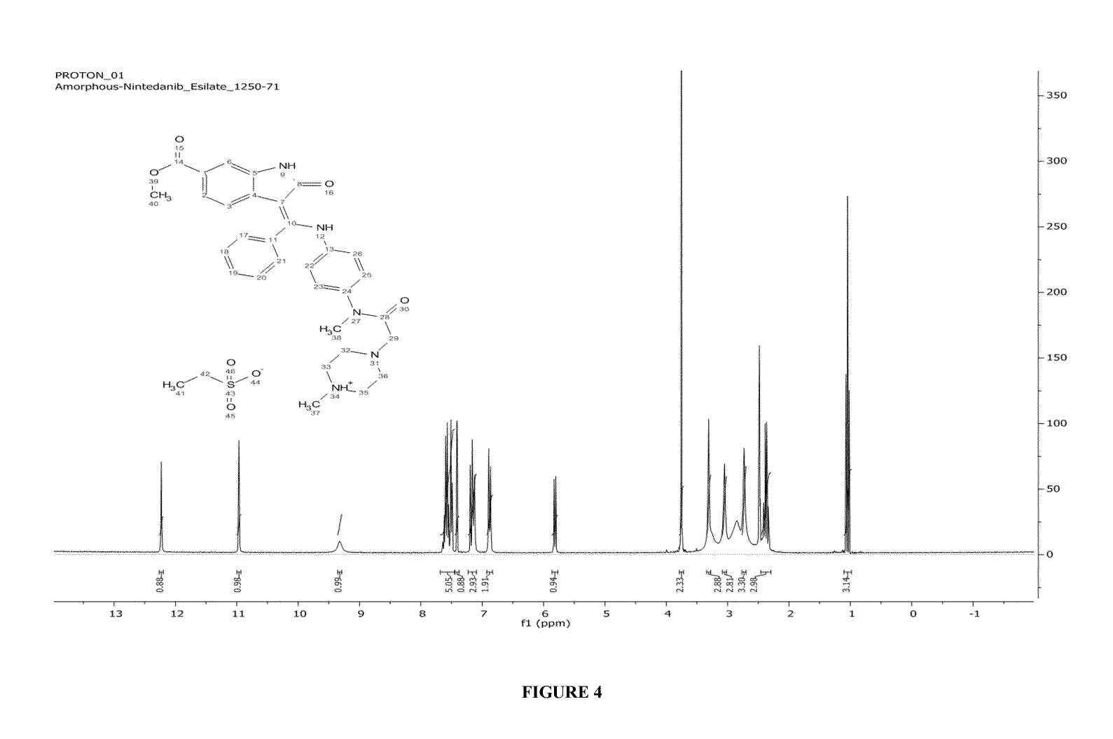

### DESCRIPTION OF THE INVENTION

A partly crystalline form of nintedanib ethanesulphonate has now been found having advantageous properties in terms of better solubility in various organic solvents and in water than the form of nintedanib ethanesulphonate hemihydrate known from WO 2004/013099.

The form of nintedanib ethanesulphonate according to the invention is obtained by dissolving nintedanib ethanesulphonate in a polar solvent, preferably water, and rapidly removing the solvent. The rapid removal of the solvent is typically obtained by freeze-drying.

For example, the form of nintedanib ethanesulphonate disclosed in WO 2004/013099 can be converted to the form according to the invention by dissolving it in water at a temperature ranging from 16° C. to 100° C., preferably from 20° C. to 70° C., and more preferably from 25° C. to 60° C. The resulting solution is then cooled rapidly to a temperature ranging from −50° C. to 0° C., preferably from −40° C. to −10° C., and more preferably from −30° C. to −20° C.

The resulting solid is maintained under vacuum for a time ranging from 0 to 72 hours, preferably from 1 hour to 48 hours, and more preferably from 2 hours to 24 hours, at a temperature ranging from −50° C. to 0° C., preferably from −40° C. to −10° C., and more preferably from −30° C. to −20° C.

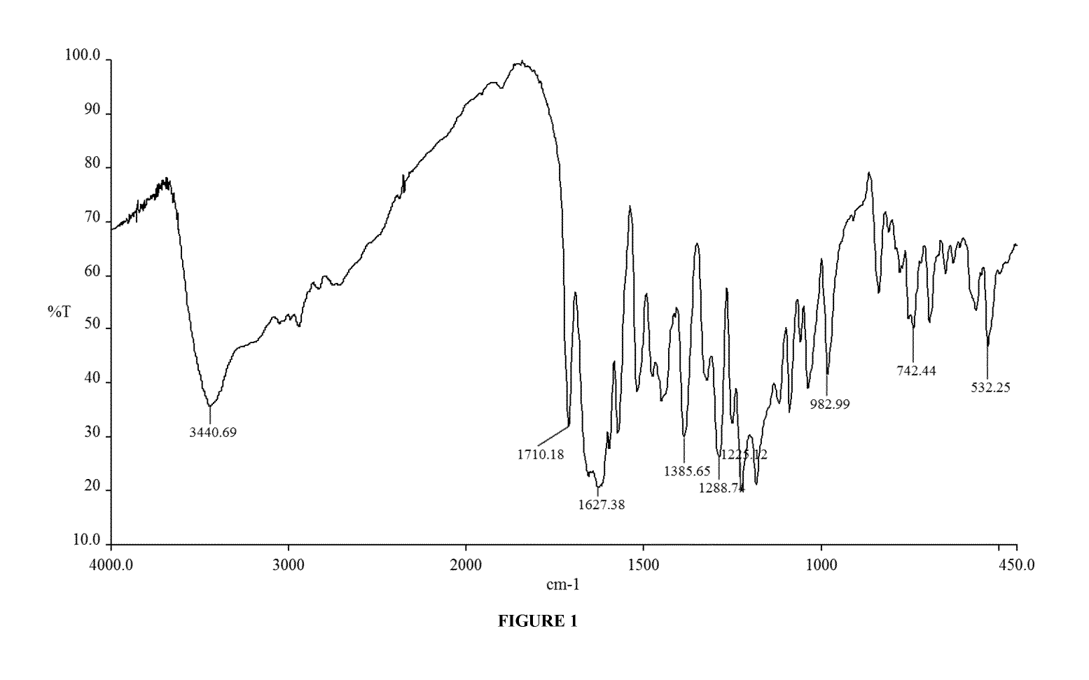

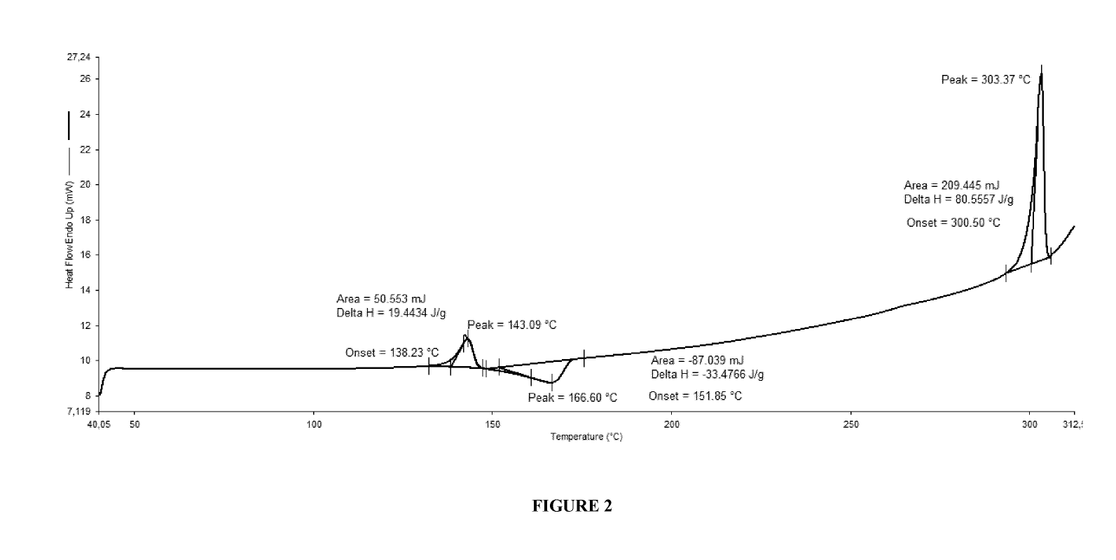

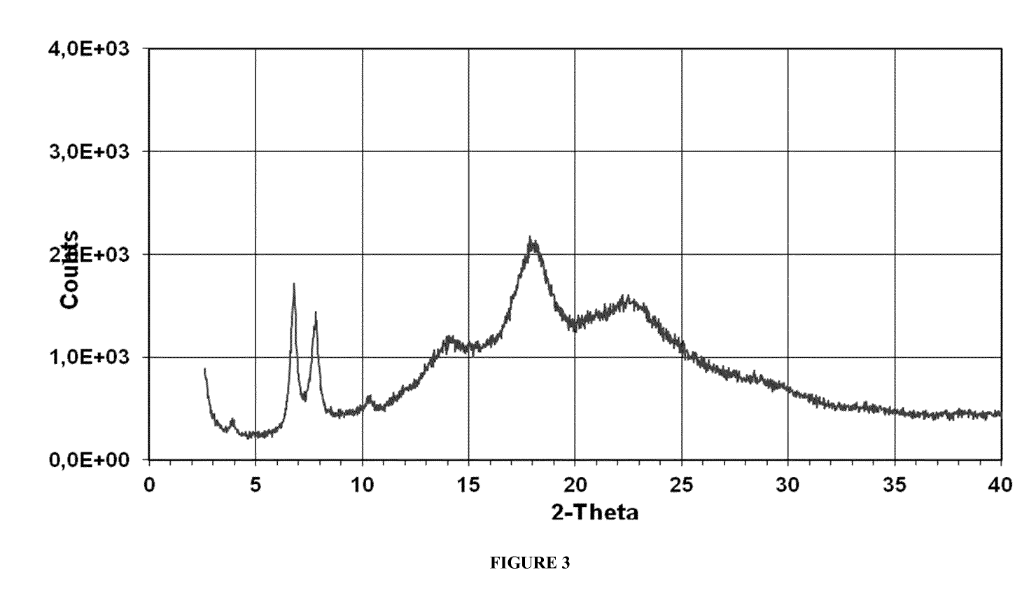

The resulting solid is a novel form of nintedanib, which has an IR spectrum, DSC curve and XRPD pattern as shown in FIGS. 1, 2 and 3 respectively.

In particular, the novel partly crystalline form of nintedanib ethanesulphonate shows:
an IR spectrum comprising absorption peaks at 3440, 1710, 1627, 1385, 1288 and 1225±1.5 cm⁻¹;
2θ peaks at 6.8, 7.8 and 17.9 in the XRPD pattern (λ=1.5418 Å);
a DSC curve comprising an exothermic peak at 143° C. and an endothermic peak at 167° C.

The invention is described in greater detail in the example below.

The IR spectra were recorded with a Perkin Elmer Spectrum 1000 IR instrument, sample preparation as KBr pellet. The spectrum is recorded by performing 16 scans at a resolution of 4 cm⁻¹.

The DSC curves were recorded with a Perkin Elmer Pyris1 instrument, and 3-5 mg of material were used to prepare the samples. The scans were performed at the rate of 10° C. a minute.

The NMR spectra were recorded with a Varian Mercury 300 instrument in DMSO-d6, 300 MHz, at 25° C., 16 scans being performed, pw=45°, rel. delay=5 s. ¹H NMR (300 MHz, DMSO-d₆) δ 12.23 (s, 1H), 10.96 (s, 1H), 9.32 (s, 1H), 7.68-7.45 (m, 5H), 7.41 (d, J=1.6 Hz, 1H), 7.23-7.09 (m, 3H), 6.93-6.83 (m, 2H), 5.81 (d, J=8.2 Hz, 1H), 3.75 (s, 2H), 3.31 (s, 3H), 3.05 (s, 3H), 2.73 (s, 3H), 2.46-2.29 (m, 3H), 1.10-0.98 (m, 3H).

The XRPD spectra were recorded with a Bruker D2 instrument which uses the following parameters: CuKα wavelength (λ=1.5418 Å); energy 30 KV; stepsize: 0.02°; 2θ range: 2.6°-40°.

### EXAMPLE

Nintedanib ethanesulphonate (25.0 g) is suspended in 300 ml of water, and the suspension is heated at T=60° C. until completely dissolved. The solution is cooled at −30° C. for 25 hours.

The resulting solid is placed in a freeze-dryer at −30° C., under vacuum, and a yellow solid is obtained after 24 hours. The product (partly crystalline form) shows an IR spectrum, DSC curve and XRPD pattern as shown in FIGS. 1-3 respectively.

1. A partially crystalline form of nintedanib ethanesulphonate.

2. A partially crystalline form of nintedanib ethanesulphonate according to claim 1, wherein IR spectrum shows absorption peaks at 3440, 1710, 1627, 1385, 1288 and 1225±1.5 cm⁻¹, 2θ peaks at 6.8, 7.8 and 17.9 in the XRPD diffractogram at the CuKα wavelength, and a DSC curve shows an exothermic peak at 143° C. and an endothermic peak at 167° C.

3. A process for the preparation of the partially crystalline form of nintedanib ethanesulphonate of claim 1, said process comprising dissolving nintedanib ethanesulphonate in a polar solvent, and subsequent rapidly removing the solvent.

4. The process according to claim 3 wherein the polar solvent is water.

5. The process according to claim 4 wherein the dissolution in water is carried out at a temperature ranging from 16° C. to 100° C.

6. The process according to claim 4 wherein the solution is rapidly cooled to a temperature ranging from −50° C. to 0° C.

7. The process according to claim 5, wherein the dissolution in water is carried out at a temperature ranging from 20° C. to 70° C.

8. The process according to claim 5, wherein the dissolution in water is carried out at a temperature ranging from 25° C. to 60° C.

9. The process according to claim 6, wherein the solution is rapidly cooled at a temperature ranging from −40° C. to −10° C.

10. The process according to claim 6, wherein the solution is rapidly cooled at a temperature ranging from −30° C. to −20° C.
